(19) United States
(12) Patent Application Publication
(10) Pub. No.: US 2014/0180121 A1
(43) Pub. Date: Jun. 26, 2014

(54) ROTATIONAL IMAGING APPARATUS WITH MONOLITHIC SHAFT

(71) Applicant: VOLCANO CORPORATION, San Diego, CA (US)

(72) Inventor: Lisa Fong, La Jolla, CA (US)

(73) Assignee: VOLCANO CORPORATION, San Diego, CA (US)

(21) Appl. No.: 14/134,253

(22) Filed: Dec. 19, 2013

Related U.S. Application Data
(60) Provisional application No. 61/740,720, filed on Dec. 21, 2012.

(51) Int. Cl. A61B 5/00; A61B 8/12

(52) U.S. Cl. CPC A61B 5/6852; A61B 8/12; A61B 5/0073
USPC 600/467; 600/407

(57) ABSTRACT

The invention generally relates to a rotational imaging apparatus with a monolithic shaft and methods of use thereof. In certain aspects, the apparatus includes a rotatable monolithic hollow elongate shaft. A rotatable elongate drive member is disposed within the shaft, and a rotatable elongate electrical signal transmission member is disposed within the drive member. The apparatus further includes an imaging device, and the shaft, the drive member and the signal transmission member are coupled to the imaging device.

### CROSS-REFERENCE TO RELATED APPLICATIONS

This application claims the benefit of, and priority to, U.S. Provisional Application Ser. No. 61/740,720, filed Dec. 21, 2012, the contents of which are incorporated by reference herein in its entirety.

### FIELD OF THE INVENTION

The invention generally relates to a rotational imaging apparatus with a monolithic shaft and methods of use thereof.

### BACKGROUND

Intravascular Ultrasound (IVUS) is an important interventional diagnostic procedure for imaging atherosclerosis and other vessel diseases and defects. In the procedure, an IVUS catheter is threaded over a guidewire into a blood vessel, and images are acquired of the atherosclerotic plaque and surrounding area using ultrasonic echoes. That information is much more descriptive than information from other imaging techniques, such as angiography, which shows only a two-dimensional shadow of a vessel lumen.

There are two types of IVUS catheters commonly in use, mechanical/rotational IVUS catheters and solid state catheters. A solid state catheter (or phased array) has no rotating parts, but instead includes an array of transducer elements (for example 64 elements). In a rotational IVUS catheter, a single transducer having a piezoelectric crystal is rapidly rotated (e.g., at approximately 1800 revolutions per minute) while the transducer is intermittently excited with an electrical pulse. The excitation pulse causes the transducer to vibrate, sending out a series of transmit pulses. The transmit pulses are sent at a frequency that allows time for receipt of echo signals. The sequence of transmit pulses interspersed with receipt signals provides the ultrasound data required to reconstruct a complete cross-sectional image of a vessel.

Typically, rotational IVUS catheters have a two piece main shaft disposed within a catheter body. A transducer is attached to a distal end of the second piece of the main shaft. A drive cable is disposed within the two pieces of the main shaft and also coupled to the transducer at its distal end. A coaxial cable is disposed within the drive cable and also coupled to the transducer. The coaxial cable delivers the intermittent electrical transmit pulses to the transducer, and delivers the received electrical echo signals from the transducer to the receiver amplifier. The IVUS catheter is removably coupled to an interface module, which controls the rotation of the shaft, the drive cable, and the coaxial cable within the catheter body and contains the transmitter and receiver circuitry for the transducer.

A problem with rotational IVUS catheters is that the second piece of the two piece shaft is free floating. During rotation, that free floating second piece experiences greater vibration than the first piece of the main shaft, which causes the second piece of the shaft to rotate at a different rate that the first piece of the shaft. The two pieces of the main shaft rotating at different rates causes kinking or winding of the drive cable. Kinking or winding of the drive cable leads to non-uniform rotation of the transducer, which causes image distortion.

### SUMMARY

The invention generally provides rotational imaging apparatuses that are configured to prevent kinking or winding of a drive member in the apparatus. Aspects of the invention are accomplished by using a single monolithic shaft as opposed to a two piece shaft. Having a one-piece monolithic shaft eliminates vibration effects on the shaft and ensures uniform rotation along the length of the shaft. Uniform rotation of the shaft ensures uniform rotation of the drive member and transducer, thereby eliminating image distortion caused by non-uniform rotation of the transducer.

Apparatuses of the invention also include a rotatable drive member disposed within the monolithic shaft, and a rotatable electrical signal transmission member disposed within the drive member. The shaft, the drive member and the electrical signal transmission member are coupled to an imaging device. The apparatus may also include a fluid injection port that is operably coupled to the shaft. The injected fluid serves to eliminate the presence of air pockets around the shaft that adversely affect image quality. The fluid can also act as a lubricant.

Any imaging device known in the art may be used with apparatuses of the invention. Exemplary devices include ultrasound devices and optical coherence tomography (OCT) devices. In certain embodiments, the imaging device is an ultrasound device and the imaging device includes an ultrasound transducer. Typically, ultrasound systems rely on conventional piezoelectric transducers, built from piezoelectric ceramic (commonly referred to as the crystal) and covered by one or more matching layers (typically thin layers of epoxy composites or polymers). Two advanced transducer technologies that have shown promise for replacing conventional piezoelectric devices are the PMUT (Piezoelectric Micromachined Ultrasonic Transducer) and CMUT (Capacitive Micromachined Ultrasonic Transducer). PMUT and CMUT transducers may provide improved image quality over that provided by the conventional piezoelectric transducer.

Generally, a connector is coupled to a proximal end of the shaft and the apparatus may connect to an interface module via the connector. The interface module typically includes components necessary for rotating the shaft, the drive member and the electrical signal transmission member. Apparatuses of the invention may additionally include an elongate catheter. In those embodiments, the shaft is configured to fit within the catheter. Apparatuses of the invention are configured from insertion in a vessel lumen, and include additional features that facilitate operation within the vessel. For example, a distal end of the body may include an atraumatic tip. The atraumatic tip is configured to guide the apparatus through the vessel lumen while avoiding perforation of the lumen. Additionally, the shaft, the drive member and the signal transmission member may be flexible so that the apparatus may more easily be advanced through the vessel.

Other aspects of the invention provide methods for imaging a vessel lumen. Such methods involve providing a rotational imaging apparatus that includes a monolithic hollow elongate shaft. A rotatable drive member is disposed within the shaft, and a rotatable electrical signal transmission member is disposed within the drive member. The shaft, the drive member and the electrical signal transmission member are coupled to an imaging device. The apparatus is inserted into a vessel lumen and used to obtain image data of the vessel lumen.

### BRIEF DESCRIPTION OF THE DRAWINGS

FIG. 1A is a simplified fragmentary diagrammatic view of a rotational IVUS probe.

FIG. 1B is a diagrammatic view within the shaft. The figure shows the drive member and the electrical signal transmission member.

FIG. 2 is a simplified fragmentary diagrammatic view of an interface module and catheter for the rotational IVUS probe of FIG. 1 incorporating basic ultrasound transducer technology.

FIG. 3 shows a prior art version of a rotational IVUS probe having a two-piece shaft.

FIG. 4 shows an embodiment of a rotational IVUS probe having a monolithic one-piece shaft.

### DETAILED DESCRIPTION

The invention generally relates to a rotational imaging apparatus with a monolithic shaft and methods of use thereof. In certain aspects, the apparatus includes a rotatable monolithic hollow elongate shaft. A rotatable elongate drive member is disposed within the shaft, and a rotatable elongate electrical signal transmission member is disposed within the drive member. The apparatus further includes an imaging device, and the shaft, the drive member and the signal transmission member are coupled to the imaging device.

Typically, apparatuses of the invention are provided in the form of a catheter. It should be noted that different imaging devices and assemblies may be used with the imaging apparatus and methods of the present invention, including, but not limited to, intravascular ultrasound (IVUS) devices and optical coherence tomography (OCT) devices.

In some embodiments, the imaging device is an IVUS imaging device. The imaging device can be a pull-back type IVUS imaging device, including rotational IVUS imaging devices. IVUS imaging devices and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein in their entirety.

The catheter will typically have proximal and distal regions, and will include an imaging tip located in the distal region. Such catheters have an ability to obtain echographic images of the area surrounding the imaging tip when located in a region of interest inside the body of a patient. The catheter, and its associated electronic circuitry, will also be capable of defining the position of the catheter axis with respect to each echographic data set obtained in the region of interest.

Besides intravascular ultrasound, other types of ultrasound catheters can be made using the teachings provided herein. By way of example and not limitation, other suitable types of catheters include non-intravascular intraluminal ultrasound catheters, intracardiac echo catheters, laparoscopic, and interstitial catheters. In addition, the probe may be used in any suitable anatomy, including, but not limited to, coronary, carotid, neuro, peripheral, or venous.

In another embodiment, the imaging apparatus may include an OCT device. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable mirror, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference mirror, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

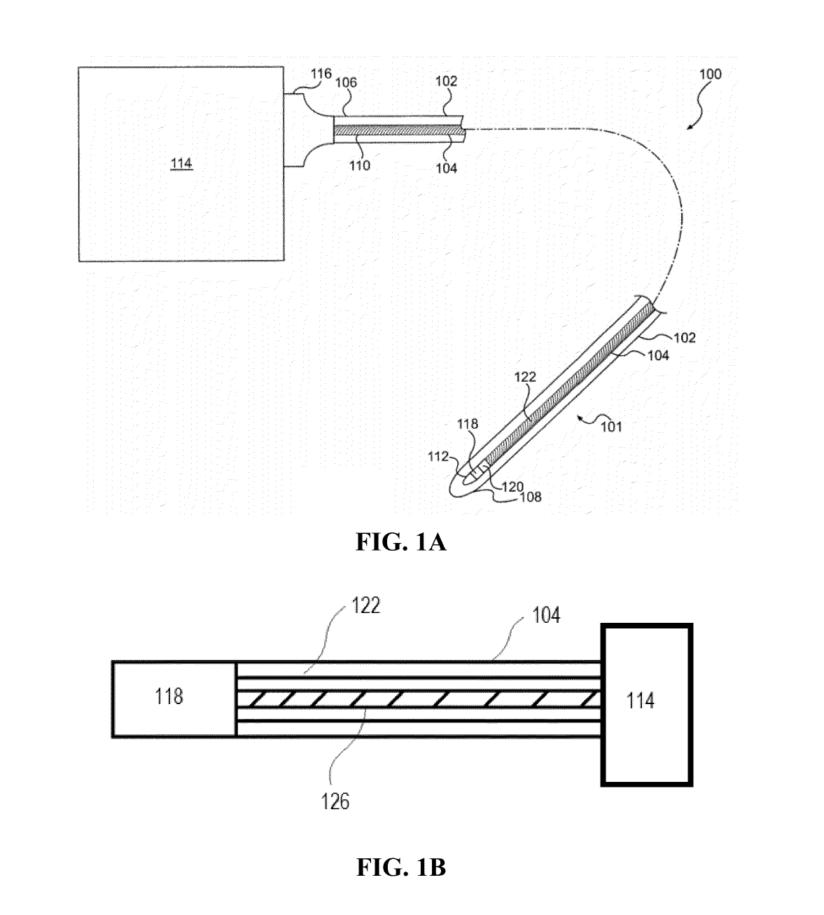

FIG. 1A shows a rotational intravascular ultrasound probe 100 for insertion into a patient for diagnostic imaging. The probe 100 includes a catheter 101 having a catheter body 102 and a hollow monolithic transducer shaft 104. The catheter body 102 is flexible and has both a proximal end portion 106 and a distal end portion 108. The catheter body 102 may be a single lumen polymer extrusion, for example, made of polyethylene (PE), although other polymers may be used. Further, the catheter body 102 may be formed of multiple grades of PE, for example, HDPE and LDPE, such that the proximal portion exhibits a higher degree of stiffness relative to the mid and distal portions of the catheter body. This configuration provides an operator with catheter handling properties required to efficiently perform the desired procedures.

The catheter body 102 is a sheath surrounding the monolithic transducer shaft 104. For explanatory purposes, the catheter body 102 in FIG. 1A is illustrated as visually transparent such that the monolithic transducer shaft 104 disposed therein can be seen, although it will be appreciated that the catheter body 102 may or may not be visually transparent.

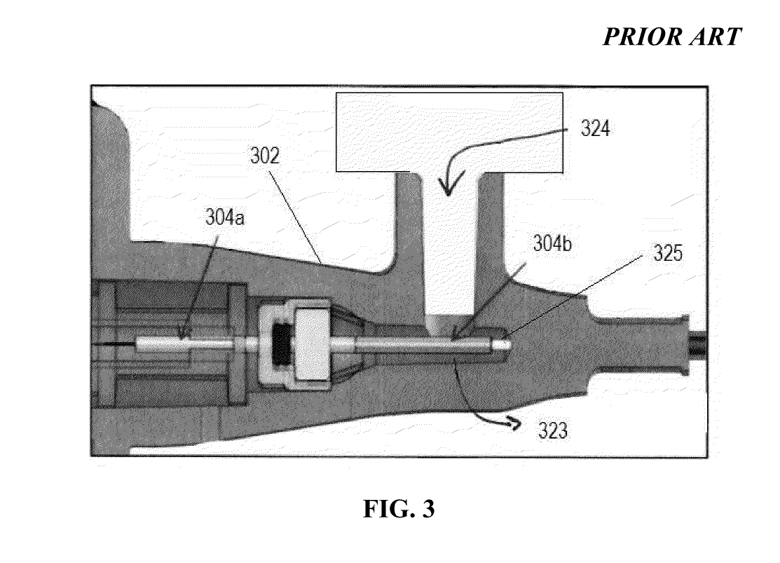

Transducer shaft 104 is a monolithic single-piece shaft, as opposed to prior art transducer shafts that are two-piece shafts. FIG. 3 illustrates a prior art rotational IVUS probe having a catheter body 302 and a two-piece shaft. In that figure, the transducer shaft has a first piece 304a and a second piece 304b. There is a space 323 between the catheter body 302 and the two-piece shaft 304a and 304b. That space provides for injection of fluid through fluid injection port 324. A drive member 305 runs coaxially through the first piece 304a and the second piece 304b. The electrical signal transmission member (not shown) runs coaxially the length of the drive member 305. In this configuration, the second piece 304b of the shaft is free floating. During rotation, that free floating second piece 304b experiences greater vibration than the first piece 304a of the shaft, which causes the second piece 304b of the shaft to rotate at a different rate that the first piece 304a of the shaft. The two pieces of the shaft rotating at different rates causes kinking or winding of the drive member 305. Kinking or winding of the drive member 305 leads to non-uniform rotation of the transducer, which causes image distortion.

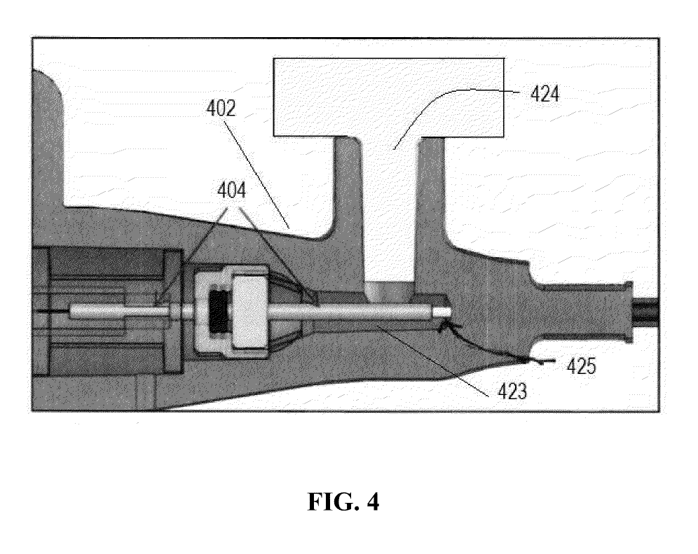

Aspects of the invention solve this problem by providing the shaft as a monolithic one-piece shaft. FIG. 4 illustrates a rotational IVUS probe having a catheter body 402 and a monolithic one-piece shaft 404. There is a space 423 between the catheter body 402 and the monolithic one-piece shaft 404. That space provides for injection of fluid through fluid injection port 424. The fluid serves to eliminate the presence of air pockets around the transducer shaft 404 that adversely affect image quality. The fluid can also act as a lubricant. A drive member 405 runs coaxially through the shaft 404. The electrical signal transmission member (not shown) runs coaxially the length of the drive member 405. Having a one-piece monolithic shaft 404 eliminates vibration effects on the shaft 404 and ensures uniform rotation along the length of the shaft 404. Uniform rotation of the shaft 404 ensures uniform rotation of the drive member 405 and transducer, thereby eliminating image distortion caused by non-uniform rotation of the transducer.

A monolithic shaft may be formed by any method known in the art. An exemplary method includes polymer extrusion of a material, for example, made of polyethylene (PE), although other polymers may be used. Further, the shaft 404 may be formed of multiple grades of PE, for example, HDPE and LDPE, such that the proximal portion exhibits a higher degree of stiffness relative to the mid and distal portions of the shaft. Other processes for producing a monolithic shaft include thermoforming. In thermoforming, a plastic sheet is heated and forced onto a mold surface. The sheet or film is heated between infrared, natural gas, or other heaters to its forming temperature, then it is stretched over or into a temperature-controlled, single-surface mold. The sheet is held against the mold surface unit until cooled, and the formed part is then trimmed from the sheet. There are several categories of thermoforming, including vacuum forming, pressure forming, twin-sheet forming, drape forming, free blowing, simple sheet bending, and the like. The monolithic shaft may also be a metal hypotube.

Referring back to FIG. 1A, The transducer shaft 104 has a proximal end portion 110 disposed within the proximal end portion 106 of the catheter body 102 and a distal end portion 112 disposed within the distal end portion 108 of the catheter body 102. The distal end portion 108 of the catheter body 102 and the distal end portion 112 of the transducer shaft 104 are inserted into a patient during the operation of the probe 100. The usable length of the probe 100 (the portion that can be inserted into a patient) can be any suitable length and can be varied depending upon the application. The distal end portion 112 of the transducer shaft 104 includes a transducer subassembly 118.

The transducer subassembly 118 is used to obtain ultrasound information from within a vessel. It will be appreciated that any suitable frequency and any suitable quantity of frequencies may be used. Exemplary frequencies range from about 5 MHz to about 80 MHz. Generally, lower frequency information (e.g., less than 40 MHz) facilitates a tissue versus blood classification scheme due to the strong frequency dependence of the backscatter coefficient of the blood. Higher frequency information (e.g., greater than 40 MHz) generally provides better resolution at the expense of poor differentiation between blood speckle and tissue, which can make it difficult to identify the lumen border. Blood speckle reduction algorithms such as motion algorithms (such as ChromaFlo, Q-Flow, etc.), temporal algorithms, harmonic signal processing, can be used to enhance images where light back scattered from blood is a problem.

The proximal end portion 106 of the catheter body 102 and the proximal end portion 110 of the transducer shaft 104 are connected to an interface module 114 (sometimes referred to as a patient interface module or PIM). The proximal end portions 106, 110 are fitted with a catheter hub 116 that is removably connected to the interface module 114.

The catheter body 102 may include a flexible atraumatic distal tip. For example, an integrated distal tip can increase the safety of the catheter by eliminating the joint between the distal tip and the catheter body. The integral tip can provide a smoother inner diameter for ease of tissue movement into a collection chamber in the tip. During manufacturing, the transition from the housing to the flexible distal tip can be finished with a polymer laminate over the material housing. No weld, crimp, or screw joint is usually required. The atraumatic distal tip permits advancing the catheter distally through the blood vessel or other body lumen while reducing any damage caused to the body lumen by the catheter. Typically, the distal tip will have a guidewire channel to permit the catheter to be guided to the target lesion over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip.

The rotation of the transducer shaft 104 within the catheter body 102 is controlled by the interface module 114, which provides a plurality of user interface controls that can be manipulated by a user. The interface module 114 also communicates with the transducer subassembly 118 by sending and receiving electrical signals to and from the transducer subassembly 118 via at least one electrical signal transmission member 126 (e.g., wires or coaxial cable) within the transducer shaft 104. The relationship of the electrical signal transmission member 126, the drive member 122, and the transducer shaft 104, is shown in FIG. 1B. The interface module 114 can receive, analyze, and/or display information received through the transducer shaft 104. It will be appreciated that any suitable functionality, controls, information processing and analysis, and display can be incorporated into the interface module 114. Further description of the interface module is provided, for example in Corl (U.S. patent application number 2010/0234736), the content of which is incorporated by reference herein in its entirety.

The transducer shaft 104 includes a transducer subassembly 118, a transducer housing 120, and a drive member 122. The transducer subassembly 118 is coupled to the transducer housing 120. The transducer housing 120 is attached to the transducer shaft 104 and the drive member 122 at the distal end portion 112 of the transducer shaft 104. The drive member 122 is rotated within the catheter body 102 via the interface module 114 to rotate the transducer housing 120 and the transducer subassembly 118. The transducer subassembly 118 can be of any suitable type, including but not limited to one or more advanced transducer technologies such as PMUT or CMUT. The transducer subassembly 118 can include either a single transducer or an array.

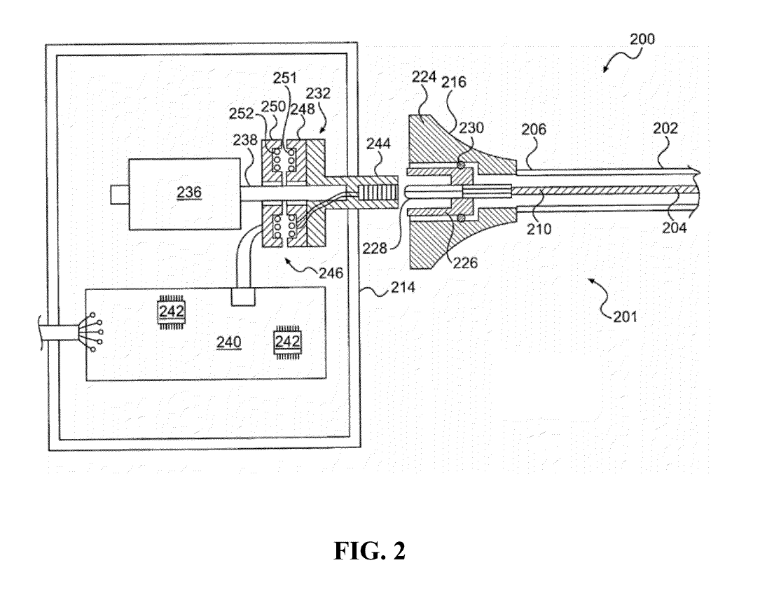

FIG. 2 shows a rotational IVUS probe 200 utilizing a common spinning element 232. The probe 200 has a catheter 201 with a catheter body 202 and a transducer shaft 204. As shown, the catheter hub 216 is near the proximal end portion 206 of the catheter body 202 and the proximal end portion 210 of the transducer shaft 204. The catheter hub 216 includes a stationary hub housing 224, a dog 226, a connector 228, and bearings 230. The dog 226 mates with a spinning element 232 for alignment of the hub 216 with the interface module 214 and torque transmission to the transducer shaft 204. The dog 226 rotates within the hub housing 224 utilizing the bearings 230. The connector 228 in this figure is coaxial. The connector 228 rotates with the spinning element 232, described further herein.

As shown, the interior of the interface module 214 includes a motor 236, a motor shaft 238, a printed circuit board (PCB) 240, the spinning element 232, and any other suitable components for the operation of the IVUS probe 200. The motor 236 is connected to the motor shaft 238 to rotate the spinning element 232. The printed circuit board 240 can have any suitable number and type of electronic components 242, including but not limited to the transmitter and the receiver for the transducer.

The spinning element 232 has a complimentary connector 244 for mating with the connector 228 on the catheter hub 216. As shown, the spinning element 232 is coupled to a rotary portion 248 of a rotary transformer 246. The rotary portion 248 of the transformer 246 passes the signals to and from a stationary portion 250 of the transformer 246. The stationary portion 250 of the transformer 246 is wired to the transmitter and receiver circuitry on the printed circuit board 240.

The transformer includes an insulating wire that is layered into an annular groove to form a two- or three-turn winding. Each of the rotary portion 250 and the stationary portion 248 has a set of windings, such as 251 and 252 respectively. Transformer performance can be improved through both minimizing the gap between the stationary portion 250 and the rotary portion 248 of the transformer 246 and also by placing the windings 251, 252 as close as possible to each other.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A rotational imaging apparatus, the apparatus comprising:
a rotatable monolithic hollow elongate shaft;
a rotatable elongate drive member within the shaft;
a rotatable elongate electrical signal transmission member within the drive member; and
an imaging device, wherein the shaft, the drive member and the signal transmission member are coupled to the imaging device.

2. The apparatus according to claim 1, further comprising a flushing port operably coupled to the shaft.

3. The apparatus according to claim 1, wherein the imaging device is coupled to a distal end of the shaft.

4. The apparatus according to claim 1, further comprising a connector coupled to a proximal end of the shaft.

5. The apparatus according to claim 4, wherein the apparatus connects to an interface module via the connector, the interface module comprising components to rotate the shaft, drive member, and the electrical signal transmission member.

6. The apparatus according to claim 1, wherein the imaging device comprises an ultrasound transducer.

7. The apparatus according to claim 6, wherein the transducer comprises a piezoelectric material.

8. The apparatus according to claim 1, wherein the shaft, the drive member, and the electrical signal transmission member are flexible.

9. The apparatus according to claim 1, further comprising an elongate catheter, wherein the shaft is configured to fit within the catheter.

10. The apparatus according to claim 1, wherein the electrical signal transmission member is coaxial cable.

11. A method of obtaining image data of a vessel lumen, the method comprising:
providing a rotational imaging apparatus that comprises a rotatable monolithic hollow elongate shaft; a rotatable elongate drive member within the shaft; a rotatable elongate electrical signal transmission member within the drive member; and an imaging device, wherein the shaft, the drive member and the signal transmission member are coupled to the imaging device; and
using the apparatus to obtain image data from within a vessel.

12. The method according to claim 1, further comprising a flushing port operably coupled to the shaft.

13. The method according to claim 11, wherein the imaging device is coupled to a distal end of the shaft.

14. The method according to claim 11, further comprising a connector coupled to a proximal end of the shaft.

15. The method according to claim 14, wherein the apparatus connects to an interface module via the connector, the interface module comprising components to rotate the shaft, drive member, and the electrical signal transmission member.

16. The method according to claim 11, wherein the imaging device comprises an ultrasound transducer.

17. The method according to claim 16, wherein the transducer comprises a piezoelectric material.

18. The method according to claim 11, wherein the shaft, the drive member, and the electrical signal transmission member are flexible.

19. The method according to claim 11, further comprising an elongate catheter, wherein the shaft is configured to fit within the catheter.

20. The method according to claim 11, wherein the electrical signal transmission member is coaxial cable.
